(19) United States
(12) Patent Application Publication
(10) Pub. No.: US 2020/0190219 A1
(43) Pub. Date: Jun. 18, 2020

(54) METHOD OF INHIBITING NONSPECIFIC BINDING TO BINDING PROTEINS THAT BIND TO SURFACE MOLECULES OF EXOSOMES OR EUKARYOTIC CELL MEMBRANES IMMOBILIZED ON CARRIER

(71) Applicant: Horiba, Ltd., Kyoto-shi, Kyoto (JP)

(72) Inventors: Shiro MIYAKE, Kyoto-shi, Kyoto (JP); Daisuke IRIKURA, Kyoto-shi, Kyoto (JP)

(21) Appl. No.: 16/630,725

(22) PCT Filed: Jul. 13, 2018
(86) PCT No.: PCT/JP2018/026518

§ 371 Date: Jan. 13, 2020

(30) Foreign Application Priority Data
Jul. 14, 2017 (JP) 2017-138155

(51) Int. Cl. C07K 17/00; G01N 33/543

(52) U.S. Cl. CPC C07K 17/00; G01N 33/54306

(57) ABSTRACT

The present invention provides a method for suppressing non-specific binding of a binding protein to a surface molecule of eukaryotic cell membrane or exosome, including immobilizing the binding protein on a carrier in the presence of gelatin.

### TECHNICAL FIELD

The present invention relates to a method for suppressing non-specific binding to a binding protein to a surface molecule of eukaryotic cell membrane or exosome immobilized on a carrier. Furthermore, the present invention relates to a method for specifically detecting the surface molecule of eukaryotic cell membrane or exosome, which method including the suppression method.

### BACKGROUND ART

Currently, the diagnosis of malignant tumors and the like is made by preliminary judgment based on image information such as visual observation, X-ray, CT (Computed Tomography), ultrasound and the like, followed by final judgment based on microscopic observation of the tissue structure using pathological tissue specimens. However, diagnosis based on such information is performed on the basis of a physician's judgment criteria, so that not a little amount of misdiagnosis may occur, which in some cases may lead to a fatal medical accident. To reduce the possibility of misdiagnosis, therefore, information on the abnormality of gene in the suspected tissue and the presence or absence of tumor markers is added, and comprehensive judgment has been made.

Tumor markers have been actively studied in recent years, and refer to tumor-related antigens, enzymes, specific proteins, metabolites, tumor genes, tumor gene products, tumor suppressor genes, and the like. For example, carcinoembryonic antigen (CEA), glycoproteins CA19-9 and CA125, prostate-specific antigen (PSA), calcitonin which is a peptide hormone produced in thyroid gland and the like have been utilized as tumor markers in some cancers for cancer diagnosis. Many tumor markers to be the detection target are body fluid (blood, lymph fluid, urine, etc.) markers, and they can be detected by known means. For example, an immunological detection method includes detection of a tumor marker by utilizing an antigen-antibody reaction. It is a detection method generally rapid, convenient and economical, as well as superior in detection accuracy. In recent years, a surface plasmon resonance (SPR) device capable of measuring a reaction and a binding amount between biomolecules of an antibody and an antigen and performing kinetic analysis by applying a surface plasmon resonance phenomenon and capturing changes in resonance angle in real time has been used in various researches and tests, and has also been applied to tumor marker tests. These methods have a great advantage in that test samples can be processed in a large amount at a low cost by immobilizing an antibody on a carrier.

However, the above-mentioned method cannot be applied to all tumor markers. Mammalian cells often have a diameter of not less than 10 μm which is extremely large for antibodies. For this reason, it is known that when a cell is brought into contact with an antibody, the cell membrane directly binds to the antibody regardless of the binding specificity of the antibody. For example, when purification of membrane surface Ig-positive cells from spleen lymphocytes is attempted using an immobilized anti-Ig antibody, the membrane surface Ig-positive cells are only about 90% of the cells that bind to the anti-Ig antibody. This indicates that the cells that bind to the anti-Ig antibody include those that bound non-specifically (non-patent document 1). Tumor markers include not only body fluid markers but also markers expressed on the surface of cancer cell membranes. When the above-mentioned method is applied to detect a tumor marker anchored on the cell membrane, a non-specific binding occurs between the cell membrane of the cell and the antibody. Thus, there has been a problem that the non-specific binding must be suppressed. However, there has been no effective method for suppressing non-specific binding of a cell membrane to an antibody.

### DOCUMENT LIST

### Non-Patent Document

non-patent document 1: Shunsuke Migita, Susumu Konda, Tasuke Honjyo, Toshiyuki Hamaoka ed., immunity experiment operation method I, II 1995. NANKODO P595

### SUMMARY OF THE INVENTION

### Problems to be Solved by the Invention

The present invention aims to provide a method for suppressing non-specific binding to a binding protein to a surface molecule of eukaryotic cell membrane or exosome immobilized on a carrier. The present invention also aims to provide a method for specifically detecting the surface molecule of eukaryotic cell membrane or exosome containing the suppression method.

### Means of Solving the Problems

The present inventors, for example, spotted an antibody (anti-c-kit antibody or negative antibody) on a biochip and immobilized same, then coated the biochip with BSA, and contacted the c-kit expressing cells with the biochip and confirmed the reflectance of the both. As a result, almost no difference in the reflectance was observed between the anti-c-kit antibody and the negative antibody. Given this result, the present inventors presumed that non-specific binding occurred between the cell membrane and both the anti-c-kit antibody and the negative antibody, thereby reducing the difference in reflectance between the two. Therefore, the present inventors conducted intensive studies in an attempt to provide a method for suppressing nonspecific binding to an antibody immobilized on a carrier, and preliminarily mixed gelatin with an antibody (anti-c-kit antibody or negative antibody) when spotting the antibody on a biochip and immobilizing same. As a result, it was found that an increase in the reflectance of the anti-c-kit antibody could be confirmed, whereas an increase in the reflectance of the negative antibody could not. In addition, the binding specificity between erythrocyte and lectin was examined under the same conditions. As a result, it was confirmed that lectin SBA, which was previously known to bind to rabbit erythrocyte, showed an increase in the reflectance, whereas lectin MAM, which was previously known to not bind to rabbit erythrocyte, did not show an increase in the reflectance. The aforementioned phenomenon confirmed by using gelatin was also observed between cells other than the above and proteins binding to the cells. Furthermore, exosome was contacted with a biochip on which antibody or lectin was immobilized under the same conditions. As a result, an increase in the reflectance of negative antibody could not be confirmed, whereas an increase in the reflectance could be confirmed in a part of lectins and antibodies. From these facts, they have found that non-specific binding to a binding protein to a surface molecule of eukaryotic cell membrane or exosome can be suppressed by using gelatin when immobilizing the binding protein on a carrier, which resulted in the completion of the present invention.

That is, the present invention provides
[1] a method for suppressing non-specific binding of a binding protein to a surface molecule of eukaryotic cell membrane or exosome, comprising immobilizing the binding protein on a carrier in the presence of gelatin;
[2] the method of [1], further comprising covering the carrier with the binding protein immobilized thereon with gelatin or casein;
[3] the method of [1] or [2], wherein the eukaryotic cell is a mammalian cell;
[4] the method of any one of [1] to [3], wherein the binding protein is an antibody or lectin;
[5] a method for specifically detecting a surface molecule of eukaryotic cell membrane or exosome, comprising (1) immobilizing a binding protein to a surface molecule of eukaryotic cell membrane or exosome on a carrier in the presence of gelatin, (2) contacting a test sample with the carrier, and (3) detecting binding between the surface molecule of eukaryotic cell membrane or exosome and the binding protein;
[6] the method of [5], further comprising covering the carrier with the binding protein immobilized thereon with gelatin or casein between step (1) and step (2);
[7] the method of [5] or [6], wherein the binding between the surface molecule of eukaryotic cell membrane or exosome and the binding protein is detected by an immunological method or a surface plasmon resonance method;
[8] the method of any one of [5] to [7], wherein the eukaryotic cell is a mammalian cell;
[9] the method of any one of [5] to [8], wherein the binding protein is an antibody or lectin;
[10] a carrier immobilized with a binding protein to a surface molecule of eukaryotic cell membrane or exosome in the presence of gelatin;
[11] the carrier of [10], wherein the carrier is further covered with gelatin or casein;
[12] the carrier of [10] or [11], wherein the eukaryotic cell is a mammalian cell;
[13] the carrier of any one of [10] to [12], wherein the binding protein is an antibody or lectin.

### Effect of the Invention

Using gelatin when immobilizing a binding protein to a surface molecule of eukaryotic cell membrane or exosome on a carrier, non-specific binding to the binding protein immobilized on the carrier can be suppressed, as a result of which binding between the surface molecule of eukaryotic cell membrane or exosome and the binding protein can be specifically detected.

### BRIEF DESCRIPTION OF THE DRAWINGS

FIG. 1 shows a Flow-cell equipped with a microarray SPRi apparatus (Horiba, Ltd.: OpenPlex).

FIG. 2 shows a biochip (Horiba, Ltd.: CS-HD) exclusive for the microarray SPRi apparatus (Horiba, Ltd.: OpenPlex). The shaded area indicates the portion where the antibody or lectin is immobilized. The hexagonal frame shows where the Gasket in FIG. 1 comes into contact.

FIG. 3 shows detection of specific binding between anti-c-Kit antibody immobilized on a biochip and c-kit on the cell membrane surface (conventional method). Respective graphs and photographs show reflectance changes (graph) and SPR images (photograph) in A P3X63Ag 8.653 cells (P3X cells; mouse myeloma cell line), B MEG01S cells (human megakaryoblast leukemia cell line), C HEK293 cells (human emboryonic kidney epithelial cell line). The graphs show the difference of the reflectance of goat IgG from the reflectance of anti-c-Kit antibody. SPR images show images 500 seconds after cell feeding.

FIG. 4 shows detection of specific binding between anti-c-Kit antibody immobilized on a biochip and c-kit on the cell membrane surface (novel immobilization method). Respective graphs and photographs show reflectance changes (graph) and SPR images (photograph) in A P3X cells, B MEG01S cells and C HEK293 cells. The graphs show the difference of the reflectance of goat IgG from the reflectance of anti-c-Kit antibody. SPR images show images 500 seconds after cell feeding.

FIG. 5 shows detection of specific binding between lectin immobilized on a biochip and rabbit erythrocyte surface sugar chain (novel immobilization method). Respective graphs and photographs show reflectance changes (graph) and SPR images (photograph) in A lectin SBA, B lectin MAM. The graphs show the difference of the reflectance of blank from the reflectance of lectin. SPR images show images 1000 seconds after cell feeding.

FIG. 6 shows detection of specific binding between each lectin or each antibody immobilized on a biochip and exosome surface sugar chain or surface antigen (novel immobilization method). Each photograph shows SPR image in each lectin (ConA; Concanavalin A, SBA; Soybean Agglutinin, MAM; Maackia amurensis, LF; Lectin, Fucose specific from Aspergillus oryzae, SSA; Lectin, sialic acid specific from Sambucus sieboldiana, AAL; Aleuria aurantia Lectin, UEA-I; Ulex europaeus Agglutinin I, Lotus; Lotus Tetragonolobus Lectin), and each antibody (CD9, CD63, CD81, Mouse IgG's). SPR images show images about 1500 seconds after diluted exosome feeding.

### DESCRIPTION OF EMBODIMENTS

The present invention provides a method for suppressing non-specific binding of a binding protein to a surface molecule of eukaryotic cell membrane or exosome, including immobilizing the binding protein on a carrier in the presence of gelatin (hereinafter sometimes to be indicated as the suppression method of the present invention).

In the suppression method of the present invention, eukaryotic cell is not particularly limited as long as it is a cell of a eucaryote and is defined as a concept including animal cell, plant cell and fungi. Among these, an animal cell and plant cell are preferable, and mammalian cell is more preferable. Examples of the mammalian cell include, but are not limited to, hepatocyte, kidney cell, splenocyte, nerve cell, glial cell, pancreatic β cell, bone marrow cell, mesangial cell, Langerhans cell, epidermal cell, epithelial cell, goblet cell, endothelial cell, smooth muscle cell, fibroblast, fiber cell, muscle cell, adipocyte, immunocyte (e.g., macrophage, T cell, B cell, natural killer cell, mast cell, neutrophil, basophil, eosinophils, monocyte), erythrocyte, megakaryoblast, megakaryocyte, synovial cell, chondrocyte, osteocyte, osteoblast, osteoclast, mammary cell, or stroma cell, and progenitor cell, stem cell, cancer cell or cultured cells thereof and the like.

Preferable examples of the plant cell include protoplasts obtained by decomposition of cell wall.

In the suppression method of the present invention, other examples of the substance having a binding molecule include galacto (ganglioside) lipid, sphingoglycolipid, membrane protein-containing organelle (e.g., chloroplast, nucleus, vesicle, rough endoplasmic reticulum, golgi apparatus, microtubule, smooth endoplasmic reticulum, mitochondria, vacuole, lysosome, centrosome) and the like.

In the suppression method of the present invention, examples of a surface molecule of cell membrane of the above-mentioned eukaryotic cell or exosome (hereinafter sometimes to be simply referred to as surface molecule) include protein, sugar chain, lipid, glycoprotein, glycolipid and the like. Among these, protein, and sugar chain are preferred. Examples of the surface protein of cell membrane or exosome include surface antigens (c-kit, CD9, CD63, CD81 and the like), and gene recombinant proteins using FC tag, clasp (Disulfide-bonded α-helical coiled-coil domains) and the like that can be constructed as a transmembrane type. Examples of the surface sugar chain of cell membrane and exosome include sugar chain antigen 125 (CA125), carcinoembryonic antigen (CEA), sialyl Tn antigen and the like.

In the suppression method of the present invention, a binding protein to a surface molecule of the above-mentioned eukaryotic cell membrane or exosome (hereinafter sometimes to be simply referred to as binding protein) is not particularly limited as long as it can specifically recognize and bind to a surface molecule of cell membrane of eukaryotic cell or exosome. For example, antibody, lectin and the like can be mentioned.

The antibody in the suppression method of the present invention encompasses both polyclonal antibody and monoclonal antibody. The antibody may encompass antibodies derived from any mammals and may belong to any of the immunoglobulin classes of IgG, IgA, IgM, IgD and IgE, preferably IgG. As the antibody, a commercially available antibody, an antibody stored in a research institute or the like that binds to the target surface molecule may also be used. Alternatively, those of ordinary skill in the art can produce an antibody according to a conventionally-known method.

In addition, the antibody includes naturally-occurring antibodies such as the aforementioned polyclonal antibody, monoclonal antibody (mAb) and the like, a chimeric antibody that can be produced using a gene recombination technique, a humanized antibody, a single-stranded antibody, and fragments of these antibodies. A fragment of an antibody means a partial region of the aforementioned antibody and specifically encompasses Fab, Fab′, F(ab′)2, scAb, scFv, scFv-Fc and the like.

In the suppression method of the present invention, lectin is not particularly limited as long as it is a sugar-binding protein or glycoprotein having a property of aggregating cells or composite carbohydrates other than antibody.

In the suppression method of the present invention, examples of the lectin that binds to surface molecule include Soybean Agglutinin (SBA), Lens culinaris Agglutinin (LCA), Aleuria aurantia Lectin (AAL), Ulex europaeus Agglutinin (UEA), Peanut Agglutinin (PNA), Wheat Germ Agglutinin (WGA), Concanavalin A (Con A), Maackia amurensis (MAM), fucose-specific lectin (LF), sialic acid-specific lectin (SSA), Lotus tetragonolobus Lectin (Lotus) and the like.

In the suppression method of the present invention, the binding protein is characterized in that it is immobilized on a carrier in the presence of gelatin. Non-specific binding to the binding protein can be suppressed by immobilizing the binding protein on a carrier together with gelatin. Suppression includes not only complete inhibition of non-specific binding, but also partial reduction. The reason why non-specific binding can be suppressed as described above is considered to be as follows. If BSA is used instead of gelatin, BSA is more hydrophobic than gelatin, so that the amount of bond water near the molecule by hydrogen bonding decreases. Therefore, it is considered that the hydrophobic portion of the protein, which is present in a large number on the cell membrane surface, is likely to bind hydrophobically to BSA, resulting in non-specific binding. Also, if the binding protein alone is immobilized, the binding protein is often more hydrophobic than gelatin, so that the protein on the cell membrane surface and the binding protein are also likely to bind hydrophobically, which leads to the occurrence of non-specific binding. On the other hand, gelatin retains a large amount of bond water in the vicinity of the molecule since it is highly hydrophilic among proteins and fibrous to allow for crossing with each other. Therefore, the presence of gelatin is considered to make it difficult for hydrophobic proteins on the cell membrane surface to bind hydrophobically. When the binding protein is immobilized on a carrier in the presence of gelatin, gelatin is present between molecules of the binding protein, and the distance between the binding proteins becomes large. That is, it is considered that a single cell is provided with a reactive surface by a binding protein rich in hydrophilicity and non-specific binding is suppressed. For the above two reasons, it is considered that non-specific binding between cells with cell membrane surfaces similarly covered with water molecules and antibody immobilized in the presence of gelatin can be suppressed. Gelatin can be prepared by producing a gelatin solution by adding a gelatin powder to heated distilled water, diluting the gelatin solution with heated distilled water to a desired concentration, and cooling same to 4° C. Immobilization of the binding protein can be performed by mixing the prepared gelatin with the above-mentioned binding protein to a final concentration of 0.005-2%, preferably 0.01-1%, and then spotting the mixture on a carrier and allowing the mixture to stand. The standing time may be appropriately determined, and may be, for example, 8 to 16 hr.

In the suppression method of the present invention, binding protein can also be immobilized on a carrier in the presence of polysaccharides instead of gelatin. Examples of the polysaccharides include agarose, agar, carrageenan, pectin, sodium alginate, glucomannan, gellan gum, xanthan gum, locust bean gum, tamarind seed gum, curdlan and the like.

The carrier to be used in the suppression method of the present invention is not particularly limited as long as it can be used for immunological method or a surface plasmon resonance method. Examples thereof include synthetic resin such as polystyrene, polyacrylamide, silicon and the like, glass, metal thin film, nitrocellulose membrane and the like.

The suppression method of the present invention may further include covering the carrier with the binding protein immobilized thereon with gelatin or casein. By further covering the carrier with gelatin, non-specific binding to a binding protein immobilized on a carrier can be further suppressed, and non-specific binding to a carrier surface part free of immobilized binding protein can also be suppressed simultaneously. Covering can be performed by adjusting the gelatin prepared according to the above-mentioned method with a solvent to a final concentration of 0.005-2%, preferably 1%, filling the surface of the carrier with the gelatin and standing same. The solvent is not particularly limited as long as it does not influence the binding between the surface molecule of eukaryotic cell membrane or exosome and the binding protein. Examples of such solvent include, but are not limited to, distilled water, PBS and the like. The time and temperature for allowing the gelatin to stand on the surface of the carrier can be appropriately determined by those skilled in the art. For example, the gelatin can be allowed to stand at room temperature for 10 min to 2 hr.

As mentioned above, non-specific binding to a binding protein can be suppressed by immobilizing the binding protein on a carrier together with gelatin. Therefore, the present invention also provides a method for specifically detecting a surface molecule of eukaryotic cell membrane or exosome, including
(1) immobilizing a binding protein to a surface molecule of eukaryotic cell membrane or exosome on a carrier in the presence of gelatin, (2) contacting a test sample with the carrier, and (3) detecting binding between the surface molecule of eukaryotic cell membrane or exosome and the binding protein (hereinafter sometimes to be also indicated as the detection method of the present invention).

In the detection method of the present invention, eukaryotic cell, surface molecule, binding protein, gelatin, and carrier may be the same as those described in the suppression method of the present invention.

In the detection method of the present invention, the test sample is not particularly limited as long as it is a sample containing or suspected of containing eukaryotic cell expressing a surface molecule of eukaryotic cell membrane or exosome to be the detection target. For example, a cell sample derived from a body fluid (blood, saliva, lacrimal fluid, urine, sweat and the like) or tissue of a subject having or suspected of having the eukaryotic cell, and the like can be mentioned.

In the detection method of the present invention, the carrier surface may be washed with a wash buffer before, after, or both before and after contacting the test sample with the carrier. The wash buffer is not particularly limited as long as it is a solvent that can suspend the test sample in the detection method of the present invention, and is a physiological salt solution suitable for a reaction between a surface molecule of a eukaryotic cell membrane or exosome and a binding protein, and an antigen-antibody reaction. Examples thereof include, but are not limited to, PBS containing 0.1% gelatin and 0.02% Tween20, and the like. The washing rate, washing time and temperature during washing of the wash buffer of the present invention can be appropriately determined by those of ordinary skill in the art.

In the detection method of the present invention, the method for detecting the binding between the surface molecule of eukaryotic cell membrane or exosome and the binding protein is not particularly limited as long as the binding can be detected. For example, an immunological method and a surface plasmon resonance method can be mentioned.

In the detection method of the present invention, the immunological method is not particularly limited as long as it is an immunological method for detecting a surface molecule of eukaryotic cell membrane or exosome—binding protein complex composed of a surface molecule of a eukaryotic cell membrane or exosome and a binding protein in a test sample by a chemical or physical means, and any measurement method may also be used. In addition, the amount of the surface molecule of eukaryotic cell membrane or exosome can also be calculated as necessary from a standard curve drawn using a standard solution containing a known amount of the surface molecule of eukaryotic cell membrane or exosome. As the immunological method, any method may be used as long as an antigen-antibody reaction is carried out on the surface of a solid phase, irrespective of a batch system or a flow system, such as ELISA and the like.

As a labeling agent used for a measurement method using a labeling substance, radioisotope, enzyme, fluorescent substance, luminescence substance and the like are used. As the radioisotope, [¹²⁵I], [¹³¹I], [³H], [¹⁴C] and the like are used.

As the above-mentioned enzyme, one which is stable and having high specific activity is preferable and, for example, β-galactosidase, β-glucosidase, alkaline phosphatase, peroxidase, malic acid dehydrogenase and the like are used. As the fluorescent substance, fluorescamine, fluorescein isothiocyanate and the like are used. As the luminescence substance, luminol, luminol derivative, luciferin, lucigenin and the like are used. In addition, a biotin-avidin system can also be used for binding an antibody and a label.

In a sandwich method, a test sample is reacted with a binding protein immobilized on a carrier (primary reaction), a labeled secondary antibody to the surface molecule of eukaryotic cell membrane or exosome is reacted (secondary reaction), and the amount (activity) of the label on the carrier is measured, whereby the surface molecule of eukaryotic cell membrane or exosome in the test sample can be detected and quantified. The primary reaction and the secondary reaction may be performed in a reverse order or performed simultaneously or at different times.

Alternatively, using an immunity sensor by a surface plasmon resonance (SPR) method, a binding molecule is immobilized on the surface of a commercially available sensor chip according to a conventional method, it is contacted with a test sample, a light with a particular wavelength is irradiated to the sensor chip from a particular angle, and the presence or absence of binding of the surface molecule of eukaryotic cell membrane or exosome to the immobilized binding protein can be determined with the change in the resonance angle as an index.

The detection method of the present invention may further include, as in the suppression method of the present invention, covering the carrier with the binding protein immobilized thereon with gelatin or casein between step (1) and step (2). Gelatin used for coating and coating method may be the same as those described in the suppression method of the present invention.

The present invention also provides a carrier immobilized with a binding protein to a surface molecule of eukaryotic cell membrane or exosome in the presence of gelatin (hereinafter sometimes to be also indicated as the carrier of the present invention). The carrier of the present invention may be further covered with the gelatin or casein.

In the carrier of the present invention, eukaryotic cell, surface molecule, binding protein, gelatin, and carrier may be the same as those described in the suppression method of the present invention.

### EXAMPLE

While the present invention is explained more specifically in the following by referring to Examples, the invention is not limited to them.

### Construction of Cell Detection Biosensor by Surface Plasmon Resonance (SPR)

A cell detection biosensor by surface plasmon resonance (SPR) was constructed using a microarray SPRi apparatus (Horiba, Ltd.: OpenPlex) and a biochip exclusive for the apparatus (Horiba, Ltd.: CS-HD; carboxy group activated by succinimide and immobilized on chip surface). The constructed sensor can measure every 3 seconds the amount of change in reflected light due to the SPR phenomenon induced by the binding of cells to the chip surface as reflectance (%). At the same time, the change of reflectance of SPR can be observed as a spot image. The chip has a surface area of 12 mm×23 mm and thus can characteristically arrange many spots in parallel by adjusting the spot diameter (spot amount) of the ligand solution for immobilization.

Comparative Example Detection of Cell with Biochip Bound with Antibody (Conventional Method: Blocking by Bovine Serum Albumin (BSA))

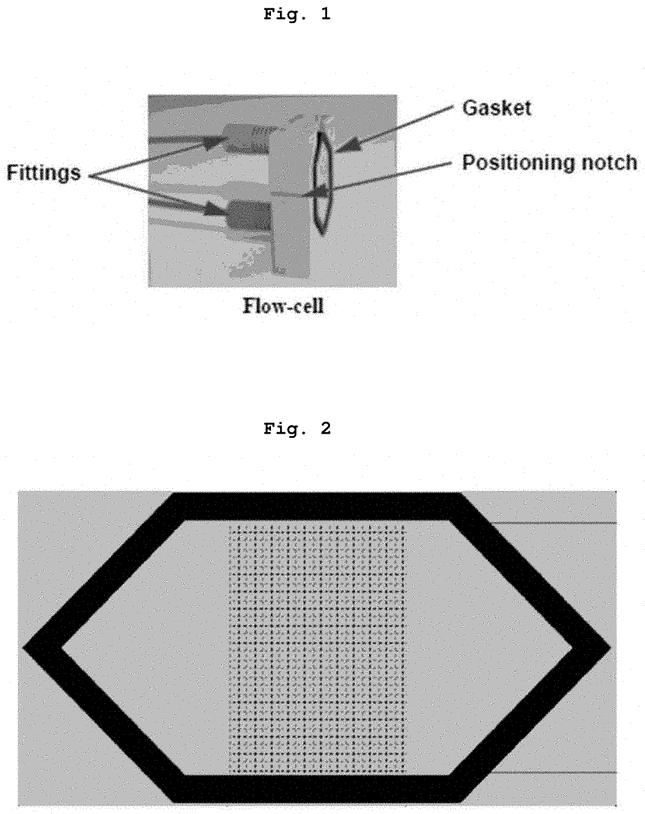

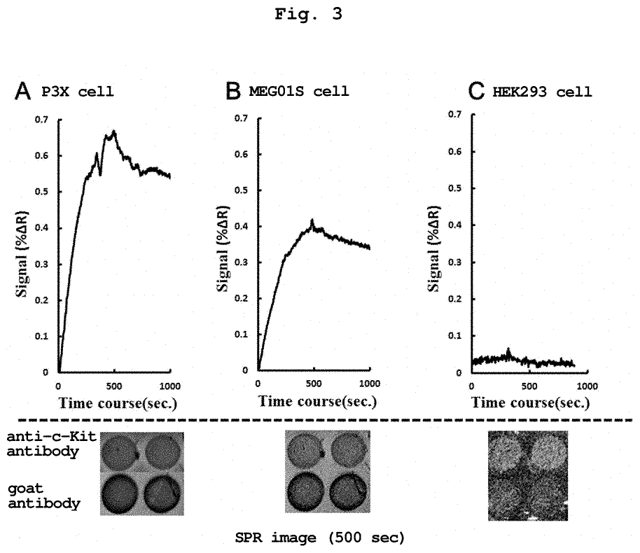

The cells used were P3X63Ag 8.653 cells (P3X cells; mouse myeloma cell line), MEG01S cells (human megakaryoblast leukemia cell line), and HEK293 cells (human embryonic kidney epithelial cell line). As the antibody, antibody (anti-c-Kit antibody; R&D systems Inc., AF1356) against c-Kit antigen (human and mouse) expressed in these cell surface was used. As the negative antibody, non-immunized goat antibody (Abcam Inc., ab37373) was used. The antibody was spotted by 10 nL on a chip surface with a spotter and immobilized by standing for 16 hr. The chip surface was washed with Dulbecco's PBS(−) (hereinafter to be abbreviated as PBS), filled with PBS containing 1% bovine serum albumin (BSA) dissolved therein, and stood for 1 hr at room temperature for blocking. The blocked chip was washed 3 times with PBS and mounted on the apparatus. The buffer or sample was contacted with the chip surface via Flow-cell (FIG. 1). Flow-cell is fixed in contact with the chip in a position (FIG. 2) where the entire Gasket is completely covered by the chip. Among the flat planes of Flow-cell, the flat plane surrounded by the frame of Gasket is recessed by 80 μm than the flat plane of the periphery of the Gasket frame. As a result, in the chip in contact with the Flow-cell, a spatial gap of 80 μm in width is generated between the flat plane surrounded by the Gasket frame of the Flow-cell and the chip surface. Therefore, a buffer or the like fed from one polyvinyl chloride tube (inner diameter 380 μm) connected to the Flow-cell via Fitting contacts the surface of the chip by filling the spatial gap of 80 μm in width, and excreted from the other polyvinyl chloride tube. PBS (buffer A) containing 0.2% BSA and 0.02% Tween 20 as a running buffer was supplied to the device equipped with the chip at a flow rate of 25 μL/min to condition the chip surface. Assuming that the reflectance at the time of stabilization was 0%, P3X cells were suspended in buffer A, fed for 480 sec, and immediately thereafter, buffer A alone was fed for 480 sec. As a result, the difference of non-immunized goat antibody reflectance from anti-c-Kit antibody reflectance was 0.67%, indicating that specific binding to the anti-c-Kit antibody could be detected. As is clear from SPRi spot image, non-specific binding was also observed in non-immunized goat antibody (FIG. 3-A). The chip was regenerated under regeneration conditions based on Yamasaki et al., AnaChem, (2016) 88, 6711-6717 (hereinafter Yamasaki et al.), MEG01S cells were also suspended in buffer A and fed for 480 sec, and immediately thereafter, buffer A alone was fed for 480 sec. As a result, the difference of non-immunized goat antibody reflectance from anti-c-Kit antibody reflectance was 0.4%, indicating that the specific binding to the anti-c-Kit antibody was detected. Similar to the results of P3X cells, non-specific binding to the non-immunized goat antibody was also observed in SPRi spot images (FIG. 3-B). HEK293 cells were also suspended in buffer A, fed for 480 sec, and immediately thereafter, buffer A alone was fed for 480 sec. As a result, the difference of non-immunized goat antibody reflectance from anti-c-Kit antibody reflectance was 0.02%, indicating that only a slight specific binding to the anti-c-Kit antibody could be detected (FIG. 3-C). As described above, a change in the reflectance was also observed in the negative antibody having no reactivity with c-kit, indicating that the negative antibody caused non-specific binding to the cell membrane of each cell. Furthermore, this indicates that the anti-c-kit antibody specifically binds to c-kit on the surface of each cell membrane and, at the same time, also nonspecifically binds to the cell membrane of each cell. The chip was blocked with BSA after immobilizing the antibody, but did not inhibit non-specific binding of the antibody to the cell membrane. Therefore, it was found that establishment of a cell membrane surface protein detection system using an antibody requires suppression of the non-specific binding.

### Example 1 Detection of Cell with Biochip Bound with Antibody (Novel Immobilization Method: Gelatin is Added During Immobilizing Antibody)

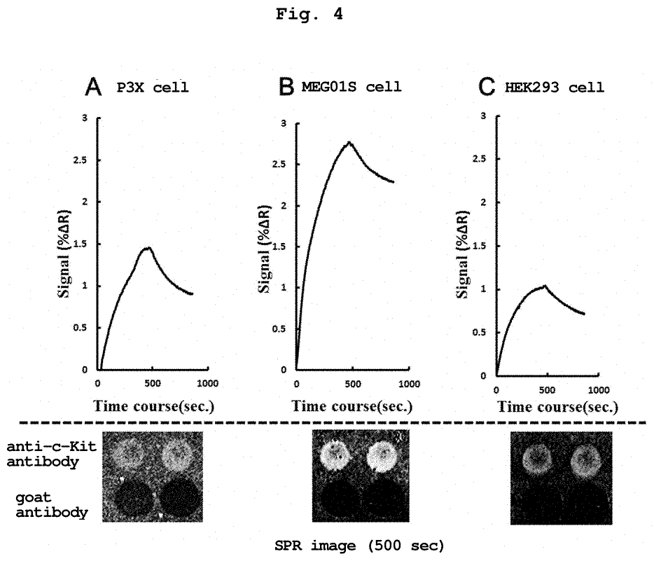

In view of the above, the inventors tried use of gelatin during immobilizing the antibody. To be specific, antibody containing 0.1% gelatin (Gelatin, fine powder (Nacalai tesque 16631-05)) was spotted by 10 nL on a chip surface of the sensor chip with a spotter and immobilized by standing for 16 hr. The chip surface was washed with PBS, filled with PBS containing 1% gelatin dissolved therein, and stood for 1 hr at room temperature for blocking. The chip was mounted on the apparatus, PBS (buffer B) containing 0.1% gelatin and 0.02% Tween 20 was fed at a flow rate of 25 μL/min for conditioning. Assuming that the reflectance at the time of stabilization was 0%, the measurement was started. P3X cells, MEG01S cells or HEK293 cells were suspended in buffer B, fed for 480 sec, changed to buffer B and further fed for 480 sec. As a result, the reflectance increased by 2.28% for P3X cells, 0.91% for MEG01S cells, and 0.72% for HEK293 cells, and the cells were specifically bound with the anti-c-Kit antibody on the chip. As a result, the specific reactivity was strikingly improved as compared to the above-mentioned experiment conditions free of gelatin (FIG. 4A-C). As shown in FIG. 4, these bonds could be easily observed in SPR spot images, and the increase in reflectance could be observed as a white image in the spot where the anti-c-Kit antibody was immobilized. On the other hand, in the negative antibody, the spot was black, and the reflectance did not increase. From these results, it was clarified that the addition of 0.1% gelatin at the time of immobilizing the antibody enabled observation of the specific interaction between the anti-c-Kit antibody and c-kit on the cell membrane.

### Example 2 Detection of Cell with Biochip Bound with Lectin (Novel Immobilization Method: Gelatin is Added During Immobilizing Lectin)

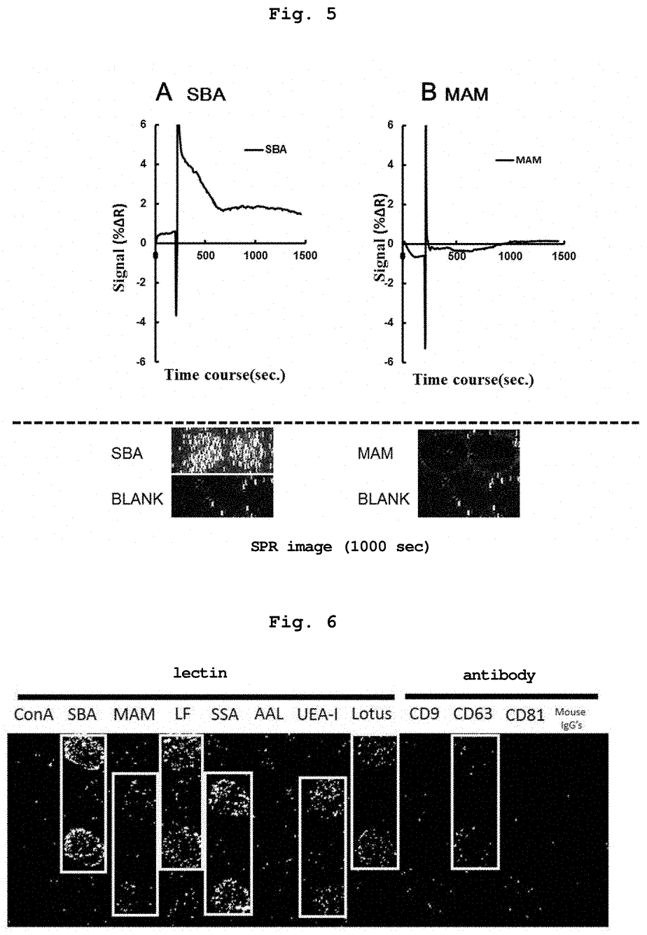

Using the constructed cell detection conditions, the binding ability of erythrocyte and lectin was examined. As the erythrocyte, EDTA-treated rabbit erythrocyte (NIPPON BIO-TEST LABORATORIES INC.) was used. As the lectin, Glycine Max (SBA), which was found to bind to rabbit erythrocyte, and Macackia amurensis (MAM), which was found to not bind to rabbit erythrocyte, were used. Similar to the antibody, lectin containing 0.1% gelatin (SBA(J117), MAM(J210); J-Oil Mills, INC.) was spotted by 10 nL on a chip surface of the sensor chip with a spotter and immobilized by standing for 16 hr. The chip surface was washed with PBS, filled with PBS containing 1% gelatin dissolved therein, and stood for 1 hr at room temperature for blocking. The chip was mounted on the apparatus, buffer B was fed at a flow rate of 25 μL/min for conditioning. Assuming that the reflectance at the time of stabilization was 0%, the measurement was started. Rabbit erythrocytes were diluted 10-fold with buffer B and fed for 240 sec under the same conditions as for the antibody. However, it was found that erythrocytes could not bind with the lectin and passed through the lectin in a continuous feeding state. Therefore, the feeding was stopped for 20 sec and the erythrocyte suspension was kept on the chip surface, whereby rabbit erythrocytes were bound to lectin. Thereafter, buffer B was fed for 1200 sec. The reflectance at this point was 1.5% for SBA, and the erythrocytes bound to immobilized SBA (FIG. 5-A). On the other hand, it did not bind to MAM (FIG. 5-B). Also in the SPR spot image, it was found that the erythrocytes were specifically bound to the immobilized SBA (FIG. 5-A, -B). From these results, it was found that the addition of 0.1% gelatin when immobilizing lectin as a ligand is effective in observing not only antibody but also specific interaction between lectin and cell.

### Example 3 Simultaneous Detection of Sugar Chain and Surface Antigen of Human Serum-Derived Exosome by SPR Image Method

Surface antigen that is membrane protein and sugar chain are present on the cell surface in addition to lipids that form cell membrane. Surface antigen is responsible for cell activation as a corresponding ligand or a receptor for outside stimulation. In addition, it is known that, after differentiation or maturation of cell by a ligand or outside stimulation, the sugar chain changes its sequence and becomes a target molecule. For example, microorganism and virus recognize a specific cell surface sugar chain and infect or invade cells. In the process of canceration of normal cells, the expression of cancer cell-specific sugar chain and the expression of specific sugar chain increase, and the surface sugar chain sequence of exosomes released by these cells also changes. Therefore, sugar chain can be expected as a useful biomarker for distinguishing microorganism, cell and exosome. In fact, in clinical settings, surface antigens and sugar chains are used as biomarkers. Surface antigens are mainly analyzed by a flow cytometer. However, sugar chain analysis has a complicated structure and is sensitively affected by many environmental factors, and analysis in a short time by a structural change or a DNA sequence is not available. Therefore, an analysis method for sugar chain is complicated and very difficult. For this reason, simultaneous detection of a surface antigen, which is a membrane protein, and sugar chain analysis has not been performed at present. In this example, therefore, simultaneous detection of sugar chain and surface antigen was performed using, as an analyte, human purified exosome assuming a human sample, and using, as a ligand, lectin that is a protein specifically recognizing a sugar chain sequence or a surface antigen-specific antibody. As a detection method, an SPRi method capable of simultaneously detecting multiple samples was used.

As human serum-derived exosomes used as analytes, purified by using Human Serum (S4200-100) (10 ml) manufactured by Biowest and exosome isolation kit PS (293-77601) manufactured by Fujifilm Wako Pure Chemical Corporation and according to the protocols thereof. As the ligand, 8 kinds of Concanavalin A (ConA; Nacalai Tesque, 09446-94), Soybean Agglutinin (SBA; J-chemical, J117), Maackia amurensis (MAM; J-chemical, J110), Aspergillus oryzae derived from purified fucose-specific lectin (LF; Tokyo Chemical Industry Co., Ltd., L0169), Sambucus sieboldiana derived from purified sialic acid-specific lectin (SSA; J-chemical, J118), Aleuria aurantia Lectin (AAL; J-chemical, J101-R), Ulex europaeus Agglutinin I (UEA-I; J-chemical, J119), Lotus tetragonolobus Lectin (Lotus; J-chemical, J109) were used for exosome sugar chain detection. In addition, 3 kinds of CD9 antibody (CD9; R&D systems Inc., MAB1880), CD63 antibody (CD63; Santa Cruz Biotechnology, sc-365604), CD81 antibody (CD81; Santa Cruz Biotechnology Inc., sc-166029), which are tetraspanin antibodies, were used for exosome surface antigen detection. As a negative control, mouse antibody (Mouse IgG's; Sigma-Aldrich Inc., 18765) was used.

0.1% Gelatin having a suppressive effect on the non-specific binding between each of the aforementioned ligands and exosome was mixed with each of the aforementioned ligands, and the mixture was spotted by 10 nL on a chip surface with a spotter and allowed to bind by standing for 16 hr. The chip surface was washed with PBS, chip surface was filled with 1% casein, and bound by standing for 16 hr at room temperature for blocking. The blocked chip was washed 3 times with PBS and mounted on the apparatus. The apparatus was fed with PBS (buffer A) containing 0.1% casein as a running buffer at a flow rate of 25 μL/min, and the reflectance at the time point of equilibration of the chip surface was taken as 0. Next, the purified exosome was diluted with buffer A to 10-fold dilution. The diluted exosome (200 μL) was injected into the apparatus and fed for 240 sec. Since the binding rate between the exosome and lectin was slow and the binding was inhibited by the flow of the liquid, the feeding was temporarily stopped, and the exosome diluted solution was kept on the chip surface for 600 sec, whereby the exosome and lectin were bound and aggregated. Thereafter, buffer A alone was further fed for 240 sec, and a total of 1080 sec was taken as the binding process. Thereafter, as a dissociation process, buffer A alone was fed for 480 sec to wash the surface of the biochip.

As a result, in the SPR image after about 1500 sec in the dissociation process by substituting with buffer A, positive lectins are SBA, MAM, LF, SSA, UEA-I, Lotus, and as for the antibody, CD63 was positive and Mouse IgG's was negative (FIG. 6). The above results simultaneously reveal that α-bound fucose and sialic acid-containing N- or O-type sugar chain and lipid-bound sugar chain are present on purified exosome, and that CD63 is present as tetraspanin, a surface antigen. In addition, since the negative control, Mouse IgG's, was negative, the measurement system was established.

### INDUSTRIAL APPLICABILITY

Non-specific binding to a binding molecule on a carrier can be suppressed using the suppression method of the present invention. The present invention has a great advantage in that test samples can be processed in a large amount at a low cost by immobilizing an antibody on a carrier. In addition, quantitative measurement of a surface molecule of eukaryotic cell membrane or exosome is possible, and an analysis method effective for industrial fields such as drug discovery, regenerative medicine, cancer diagnosis and the like can be provided. This application is based on a patent application No. 2017-138155 filed in Japan (filing date: Jul. 14, 2017), the contents of which are incorporated in full herein.

1. A method for suppressing non-specific binding of a binding protein to a surface molecule of eukaryotic cell membrane or exosome, comprising immobilizing the binding protein on a carrier in the presence of gelatin.

2. The method according to claim 1, further comprising covering the carrier with the binding protein immobilized thereon with gelatin or casein.

3. The method according to claim 1, wherein the eukaryotic cell is a mammalian cell.

4. The method according to claim 1, wherein the binding protein is an antibody or lectin.

5. A method for specifically detecting a surface molecule of eukaryotic cell membrane or exosome, comprising (1) immobilizing a binding protein to a surface molecule of eukaryotic cell membrane or exosome on a carrier in the presence of gelatin, (2) contacting a test sample with the carrier, and (3) detecting binding between the surface molecule of eukaryotic cell membrane or exosome and the binding protein.

6. The method according to claim 5, further comprising covering the carrier with the binding protein immobilized thereon with gelatin or casein between step (1) and step (2).

7. The method according to claim 5, wherein the binding between the surface molecule of eukaryotic cell membrane or exosome and the binding protein is detected by an immunological method or a surface plasmon resonance method.

8. The method according to claim 5, wherein the eukaryotic cell is a mammalian cell.

9. The method according to claim 5, wherein the binding protein is an antibody or lectin.

10. A carrier immobilized with a binding protein to a surface molecule of eukaryotic cell membrane or exosome in the presence of gelatin.

11. The carrier according to claim 10, wherein the carrier is further covered with gelatin or casein.

12. The carrier according to claim 10, wherein the eukaryotic cell is a mammalian cell.

13. The carrier according to claim 10, wherein the binding protein is an antibody or lectin.
